(19) United States
(12) Patent Application Publication
(10) Pub. No.: US 2010/0120162 A1
(43) Pub. Date: May 13, 2010

(54) PROCESS FOR DETERMINING SULFUR CONTENT IN FUEL

(75) Inventor: Ralf Stich, Buchenbach (DE)

Correspondence Address: HOFFMANN & BARON, LLP, 6900 JERICHO TURNPIKE, SYOSSET, NY, 11791, US

(73) Assignee: TESTO AG, Lenzkirch (DE)

(21) Appl. No.: 12/267,933

(22) Filed: Nov. 10, 2008

(51) Int. Cl. G01N 31/12; G01N 33/00

(52) U.S. Cl. 436/119; 422/94

(57) ABSTRACT

With a method for the determination of the sulfur content in fuels, a fuel sample is introduced into a miniaturized and/or microstructured combustion chamber (2) for thermal oxidation of the total sulfur, wherein an electrochemical gas sensor (3) is provided for the determination of the SO₂ content in the gas produced during the thermal oxidation, and gas transport to the gas sensor (3) is brought about by a pump (4). The thermal oxidation takes place hereby by a pyrolysis in the micromechanically produced combustion chamber (2), wherein the energy for the thermal oxidation is preferably supplied via an electric heating platform or a heating wire.

### BACKGROUND OF THE INVENTION

The invention concerns a method for the determination of the sulfur content in fuels and a device to determine the sulfur content of a fuel sample.

In the emission of climate-damaging SO₂, the sulfur content in fuels, such as diesel, heavy oil, and heating oil plays an important role. For this reason, the maximum limits for sulfur in diesel fuels and heating oil have continued to be lowered in the last few years. It is to be expected in the future that the maximum limits for the sulfur content in heavy oil used as fuel for shipping, for example, under the MARPOL guidelines, must be clearly lowered. In order to maintain the maximum limits, the need for measurement technology for the determination of the sulfur content will clearly increase in the future.

The analysis of the sulfur content of an oil sample in a laboratory is known.

Up to now, for example, the following measurement methods have been used: A total sulfur content measurement in petroleum chemistry within the framework of a process monitoring takes place using X-ray fluorescence spectrometry or liquid chromatography. The advantages of this method lie in that an online measurement is possible, low detection limits can be implemented, and a high selectivity is attainable. The disadvantage in this method is that its execution is expensive and trained personnel are necessary.

In laboratory analysis, chromatographs or IR spectrometers or UV spectrometers are used to measure SO₂ produced by thermal oxidation. The disadvantage hereby is the high power consumption by the thermal oxidation of the oil sample in a furnace and the need for additional media, for example, pure oxygen, and the comparatively large expense with the use of a spectroscopic detection method.

DE 197 41 809 B4 discloses a method for total sulfur determination by the pyrolysis of sulfur-containing compounds, together with methanol, at elevated temperatures in the range of 1200° C., preferably between 400 and 800° C., and the determination of the H₂S. The detection of H₂S takes place, either directly by the capturing in an absorption solution, by microcoulometric titration, with the aid of a flame-photometric detector or by chemoluminescence.

### SUMMARY OF THE INVENTION

The goal of the invention is to prepare a simple-to-manage method for the determination of the sulfur content and a device to carry out this method.

To attain this goal, the method in accordance with the invention for the determination of the sulfur content in fuels provides for the introduction of a sample of a fuel to be investigated in a combustion chamber, for the sulfur contained in the sample to be oxidized to SO₂, for the gas mixture formed in the combustion chamber after the oxidation of the sulfur, or a part thereof, to be conducted to a gas sensor sensitive to SO₂ in the gas mixture, for the SO₂ fraction of the gas mixture to be ascertained by means of the gas sensor and for the sulfur content of the sample to be determined from the ascertained SO₂ fraction of the gas mixture. Preferably, the combustion chamber is miniaturized and/or designed to be in a microstructured form. Thus, a method is described which can be implemented with simple components, wherein a simple management is possible when carrying out the method.

It is particularly favorable thereby if the gas sensor is an electrochemical SO₂ sensor. Electrochemical SO₂ sensors have proved good, in actual practice, when used under rough environmental conditions and thus guarantee a method execution feasibility which is not susceptible to disturbances, even with untrained personnel.

In accordance with one configuration of the invention, provisions can be made so that the oxidation of the sulfur takes place by thermal oxidation, in particular, complete thermal oxidation. It is advantageous that the conversion of the fuel sample into, among other things, SO₂, can be executed in a combustion chamber which is designed to be relatively small.

A particularly simple loading of the combustion chamber can be carried out if the fuel sample is present in liquid form, in particular, as an oil.

A particularly simple method results if, during the thermal oxidation, the atmospheric oxygen in the combustion chamber is used. Thus, the supply of other substances promoting oxidation in the combustion chamber can be omitted, wherein the apparatus outlay for the execution of the method is kept relatively small.

Support of thermal oxidation can be accomplished through the supply of a fuel gas. It is thereby advantageous that the oxidation can thus be carried out more rapidly and/or with a lower electrical expense.

The goal is also attained with a device for the determination of the sulfur content in a fuel sample in which a combustion chamber to hold the fuel sample is present; the combustion chamber has a means for the thermal oxidation of the fuel sample; and a gas sensor which is sensitive to SO₂ can be provided with gases from the combustion chamber. Preferably, the combustion chamber is miniaturized and/or constructed in a microstructured form and designed for the thermal oxidation of the total sulfur in the fuel sample. Thus, a device is made available by which the method in accordance with the invention can be carried out. This device can therefore be preferably implemented with a low-cost structure, which comprises a simple-to-manage combustion chamber and inexpensive gas sensory device.

An electrochemical SO₂ sensor and/or a semiconductor gas sensor can be used as the gas sensor, wherein the use of semiconductor gas sensors bring about an increased cross sensitivity with respect to the accompanying gases NO₂, O₂, and the like.

To supply the measurement gas produced from the oxidation of the fuel sample to the gas sensor, a pump may be provided so that the gas sensor can be supplied with gases from the combustion chamber by means of the pump. Thus, a device is preferably designed that makes available, quickly and reliably, the desired measurement values for the sulfur content in the fuel sample.

Alternatively, a design is possible where the gas sensor can be supplied, by means of diffusion, with gases from the combustion chamber. This diffusion can be supported by a slight excess pressure, forming during oxidation. With this configuration, it is advantageous that a pump can be omitted along with the energy necessary for this, wherein the construction expense and the operating expense of the device are once again reduced.

An advantageous arrangement of the components of the device is produced if the gas sensor is placed in the gas flow direction between the combustion chamber and the pump. Thus, the pump is advantageously provided downstream from the combustion chamber, wherein the measurement gases forming during the oxidation and to be investigated can be readily brought into the measurement area of the gas sensor.

A tube and/or hose system is preferably provided to conduct the gas flow between the individual components.

For the controlled supply of the measurement gas forming during the oxidation, a cutoff valve may be provided in the gas flow direction between the combustion chamber and the gas sensor. This cutoff valve can be closed during the oxidation process and can be opened only after conclusion of the oxidation, wherein the forming measurement gas can flow, controlled, to the gas sensor and can be evaluated in a defined manner. Thus, the accuracy of the sulfur content determination is increased even more.

To increase the defined reaction of the fuel sample during the oxidation, the combustion chamber can be provided an airtight opening which can be closed. After filling the combustion chamber with a fuel sample, this opening can be closed, wherein during the thermal oxidation, no fraction of the measurement gas being formed is lost.

A simple-to-manage device results if the combustion chamber can be heated electrically. This allows, advantageously, for the oxidation to be initiated and controlled electrically, which facilitates the carrying out of the method. Furthermore, additional fuels for the oxidation of a fuel sample can be omitted.

A particularly compact configuration of the device in accordance with the invention results if the combustion chamber is designed as a micropyrolysis chamber. This combustion chamber is preferably produced micromechanically.

In accordance with an advantageous configuration of the invention, the device can be designed to be portable. Thus, the device can be used simply for sulfur content determination on or at vehicles, for example, land vehicles, ships, or planes.

To improve the feasibility of the thermal oxidation, a supply container with a fuel gas may be provided, from which the combustion chamber can be filled. Advantageously, a microburner can be provided for the oxidation bringing about the complete combustion of the oil sample instead of or in addition to an electrically heated combustion chamber.

To obtain an accurate and comparable measurement value for the sulfur content of the fuel sample, a metering unit may be provided to fill the combustion chamber with a defined fuel sample quantity, in particular, in liquid form and/or as an oil sample. As a metering unit, for example, a pipette or a spray nozzle or another means can be used to introduce a predetermined quantity of fuel sample into the combustion chamber. The use of a metering unit advantageously makes possible the determination of the mass and/or volume fraction of sulfur per volume unit mass and/or of the investigated fuel.

In accordance with a configuration of the invention, a means is present for the determination of the sulfur content of the fuel sample from the sensor signals of the gas sensor. Advantageously, a display means to show the sulfur content is provided. This gives the advantage that, by indicating the sulfur content directly, conclusions can be reached as to the degree of environmental damage which could be produced by using the investigated fuel.

To attain a defined and/or complete thermal oxidation in the combustion chamber, provisions can be made so that a means is provided to recognize complete pyrolysis of the fuel sample in the combustion chamber. Such means can be, for example, a means for thermal monitoring of the oxidation process in the combustion chamber, for optical monitoring, and/or a means for the mass determination of the fuel sample, and/or another means. This gives the advantage that, by monitoring the oxidation process, measurement error samples can be avoided, in particular, systematic measurement errors resulting from incomplete thermal oxidation of the fuel.

The invention will now be described with the aid of embodiment examples, but is not limited to the embodiment examples. Other embodiment examples may result through a combination of the features of the patent claims with one another and/or with features of the embodiment examples.

### BRIEF DESCRIPTION OF THE DRAWINGS

The figures show the following.

FIG. 1: the schematic structure of a device in accordance with the invention for the determination of the sulfur content of a fuel sample;

FIG. 2: the schematic structure of another device for the determination of the sulfur content of a fuel sample; and

FIG. 3: the schematic structure of a third device for the determination of the sulfur content of a fuel sample.

### DETAILED DESCRIPTION OF THE INVENTION

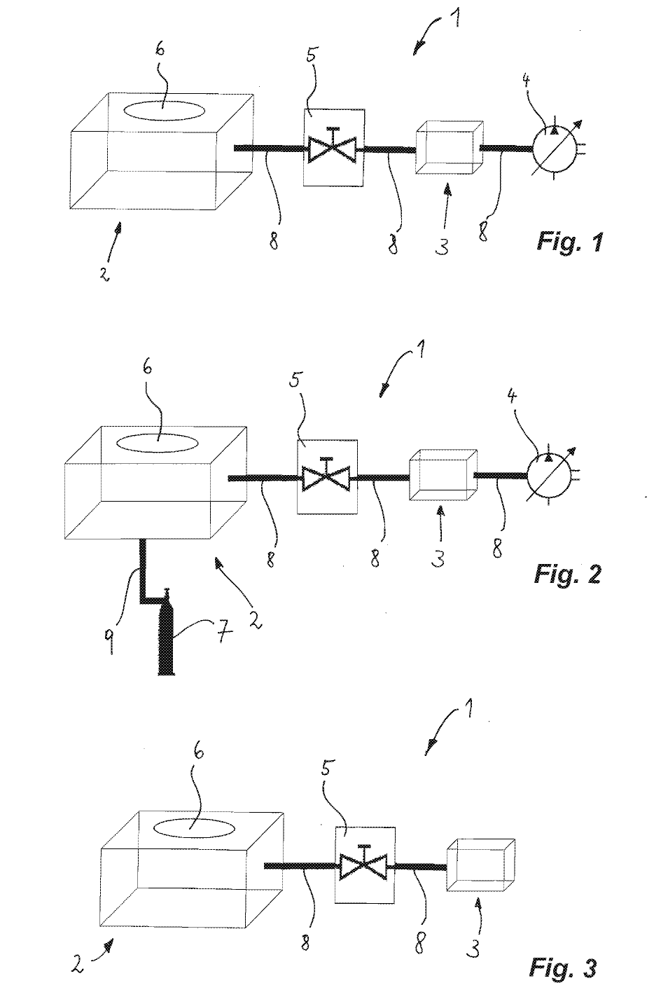

FIG. 1 shows a sulfur content measurement device that is designated, as a whole, with 1 and has a combustion chamber 2 to hold a fuel sample, wherein the combustion chamber 2 has a means for the thermal oxidation of the fuel sample. This means, not illustrated in FIG. 1, is designed as an electrically heatable micropyrolysis chamber. To detect the SO₂ that forms during thermal oxidation of the fuel sample in the combustion chamber 2, a gas sensor 3 is provided that is designed, in the embodiment example, as an electrochemical SO₂ sensor.

To provide the gas sensor 3 with the gases produced in the combustion chamber 2 by thermal oxidation of the fuel sample, a pump 4 is provided, wherein the gas sensor 3 is placed in the gas flow direction between the combustion chamber 2 and pump 4.

A cutoff valve 5 is provided between the combustion chamber 2 and the gas sensor 3; with this valve, the gas flow between the combustion chamber 2 and gas sensor 3 can be cut off.

The combustion chamber 2 has an opening 6, which can be closed airtight with a lid, through which the fuel sample can be introduced into the combustion chamber 2. Pump 4, gas sensor 3, cutoff valve 5, and combustion chamber 2 are connected by a tube system 8, by means of which the gas produced by thermal oxidation of the fuel sample in the combustion chamber 2 flows. This gas flow is driven by pump 4 in the embodiment example in accordance with FIG. 1.

The sulfur content measurement device 1 shown in the schematic representation according to FIG. 1 is located in a common housing, not illustrated, and designed to be portable.

In another embodiment example, the gas flow is driven from the combustion chamber 2 to the gas sensor 3 by diffusion, wherein pump 4 in FIG. 1 is omitted. The diffusion is supported hereby by excess pressure produced during the oxidation.

In this embodiment example also, the combustion chamber 2, the cutoff valve 5, and the gas sensor 3 are connected by a tube system 8.

In general, the tube system 8 can also be replaced by a hose system.

In the embodiment example in accordance with FIG. 2, the sulfur content measurement device, designated 1, comprises, in addition to the configuration in accordance with FIG. 1, a supply container 7 for the gas provisioning of the combustion chamber 2 with combustion gas, for example, H₂.

The combustion chamber 2 is designed as a microcombustion chamber.

Gas sensor 3 may be an electrochemical SO₂ sensor or an SO₂ semiconductor sensor.

The sulfur content measurement device 1, shown in FIG. 3, differs from the embodiment examples in accordance with FIG. 1 and FIG. 2 in that a pump is not present. The sulfur content measurement device 1 therefore has a combustion chamber 2 to hold a fuel sample, an electrochemical gas sensor 3 for the detection of SO₂ and for the determination of the concentration of SO₂, and a cutoff valve 5, located between these components.

The combustion chamber 2, the cutoff valve 5, and the gas sensor 3 are connected, as in the preceding embodiment examples, by a tube system 8. The combustion chamber 2, which, again, is designed as a microcombustion chamber, also has an opening 6 that can be closed airtight in this embodiment example. After introduction of the fuel sample into the combustion chamber 2, which can be closed airtight, the fuel sample is thermally oxidized in the combustion chamber, wherein the energy is supplied for the thermal oxidation via an electric heating platform, which is not given more detail.

By means of the gas formed during the thermal oxidation, an excess pressure arises in the combustion chamber 2, which is sufficient to convey a fraction of the gas produced through the tube system 8, with the cutoff valve open, to the gas sensor 3.

Determination of the concentration of the SO₂ fraction in the gas produced takes place in the gas sensor 3, from which it is possible to calculate the sulfur content in the fuel sample with a known fuel sample quantity.

With the sulfur content measurement device 1 in accordance with FIG. 1, it is possible to carry out a method in accordance with the invention for the determination of the sulfur content in fuels; this is described in more detail below.

First, a fuel sample of a fuel to be investigated is introduced into the combustion chamber 2, wherein the cutoff valve 5 is closed and the pump 4 is turned off.

Then, the opening 6, which can be closed airtight, is closed.

Subsequently, the total fuel sample is completely oxidized thermally with the atmospheric oxygen found in the combustion chamber. The fuel sample is hereby reacted to H₂O, CO₂, NO₂, SO₂, O₂ and other components.

Then, the cutoff valve 5 is opened, and the pump 4 is started. In this way, the gas produced by thermal oxidation arrives, with the aforementioned components, at the gas sensor 3.

The SO₂ content in the gas produced can then be measured with the gas sensor 3. The total sulfur content of the sample results from the determined SO₂ content, since the total sulfur content correlates with the SO₂ content.

With the sulfur content measurement device 1 in accordance with FIG. 2, it is possible to carry out a method to determine the sulfur content in fuels, which is described in more detail below.

First, a fuel sample of a fuel to be investigated is introduced into the combustion chamber 2.

Subsequently, the opening, which can be closed airtight, is closed, the cutoff valve 5 is blocked, and the pump 4 is turned off.

Then, via the supply line 9 from the supply container 7, a fuel, for example, H₂, is conducted into the combustion chamber 2, and the total fuel sample found in the combustion chamber 2 is burned. To this end, the fuel is ignited.

The combustion takes place using the atmospheric oxygen enclosed with the fuel sample in the combustion chamber 2.

After a complete burning of the fuel sample, the cutoff valve 5 is opened; the pump 4 is started; and the SO₂ content is measured with the gas sensor 3. In turn, the total sulfur content of the sample is found from the SO₂ content.

With the sulfur content measurement device 1 in accordance with FIG. 3, a method can be carried out to determine the sulfur content in fuels, which, except for the starting of a pump, proceeds in the same way as the method described in FIG. 1.

In the method for the determination of the sulfur content in fuels, a fuel sample is introduced into a miniaturized and/or microstructured combustion chamber 2 for the thermal oxidation of the total sulfur, wherein an electrochemical gas sensor 3 is provided for the determination for the SO₂ content in the gas produced during the thermal oxidation, and gas transport to the gas sensor 3 is brought about by a pump 4. The thermal oxidation takes place here by a pyrolysis in the micromechanically produced combustion chamber 2, wherein the energy for the thermal oxidation is preferably supplied via an electric heating platform or a heating wire.

1. Method for the determination of the sulfur content in fuels, characterized in that a fuel sample of a fuel to be investigated is introduced into a combustion chamber (2); that the sulfur contained in the sample is oxidized by thermal oxidation to SO₂ in the combustion chamber (2) to form a gas mixture; that after the oxidation of the sulfur, the gas mixture formed in the combustion chamber (2), or a part thereof, is conducted to a gas sensor (3), which is sensitive to SO₂ in the gas mixture; that the SO₂ fraction of the gas mixture is ascertained by means of the gas sensor (3); and that the sulfur content of the sample is determined from the ascertained SO₂ fraction of the gas mixture.

2. Method according to claim 1, characterized in that the gas sensor (3) is an electrochemical SO₂ sensor.

3. (canceled)

4. Method according to claim 1, characterized in that the fuel sample is introduced into the combustion chamber (2) in liquid form.

5. Method according to claim 1, characterized in that atmospheric oxygen in the combustion chamber (2) is used during the oxidation.

6. Method according to claim 1, characterized in that the oxidation is supported by addition of a fuel gas to the combustion chamber (2).

7. Device for the determination of the sulfur content of a fuel sample, characterized in that a combustion chamber (2) is present to hold the fuel sample; that the combustion chamber (2) has a means for the thermal oxidation of the fuel sample; and that a gas sensor (3), which is sensitive to SO₂ is provided to detect SO₂ levels of gases from the combustion chamber (2).

8. Device according to claim 7, characterized in that a pump (4) is present and that the gas sensor (3) is provided with gases from the combustion chamber (2) by means of the pump (4).

9. Device according to claim 8, characterized in that the gas sensor (3) is located in the gas flow direction between the combustion chamber (2) and the pump (4).

10. Device according to claim 9, characterized in that a cutoff valve (5) is provided in the gas flow direction between the combustion chamber (2) and the gas sensor (3).

11. Device according to claim 7, characterized in that the gas sensor (3) can be supplied with gases from the combustion chamber (2) by means of diffusion.

12. Device according to claim 7, characterized in that the gas sensor (3) is an electrochemical SO₂ sensor.

13. Device according to claim 7, characterized in that the combustion chamber (2) has an opening (6), which can be closed airtight.

14. Device according to claim 7, characterized in that the combustion chamber can be heated electrically.

15. Device according to claim 7, characterized in that the combustion chamber is designed as a micropyrolysis chamber.

16. Device according to claim 7, characterized in that the device (1) is portable.

17. Device according to claim 7, characterized in that a means for the determination of the sulfur content of the fuel sample from the sensor signals of the gas sensor (3) is present.

18. Device according to claim 7, characterized in that a means is provided to recognize the complete pyrolysis of the fuel sample in the combustion chamber (2).

19. An assembly comprising:
a device according to claim 7; and,
a supply container (7) provided with the fuel gas, from which the combustion chamber (2) can be filled.

20. Assembly according to claim 19, characterized in that a metering unit is provided to fill the combustion chamber (2) with a defined fuel sample quantity.
